(19) United States
(12) Patent Application Publication
(10) Pub. No.: US 2024/0099571 A1
(43) Pub. Date: Mar. 28, 2024

(54) LARYNGOSCOPE WITH A PLURALITY OF LIGHT EMITTING COMPONENTS ARRANGED IN AN ARC SHAPE

(71) Applicant: AnesTek Corp., Taoyuan City (TW)

(72) Inventor: Chih-Yu Ting, Taoyuan City (TW)

(73) Assignee: AnesTek Corp., Taoyuan City (TW)

(21) Appl. No.: 18/097,499

(22) Filed: Jan. 17, 2023

Related U.S. Application Data
(60) Provisional application No. 63/410,606, filed on Sep. 27, 2022.

(30) Foreign Application Priority Data
Nov. 8, 2022 (TW) 111142609

(51) Int. Cl. A61B 1/267; A61B 1/00; A61B 1/04; A61B 1/06

(52) U.S. Cl. CPC A61B 1/267; A61B 1/00052; A61B 1/00154; A61B 1/04; A61B 1/0661

(57) ABSTRACT

A laryngoscope is provided and includes a handle, a blade assembly and a plurality of light emitting components. The blade assembly includes a supporting body and a sleeve body. The supporting body is pivotally connected to the handle. The sleeve body is sleeved outside the supporting body. The plurality of light emitting components are disposed on the supporting body and arranged in an arc shape on a first flat plane. Besides, at least one light divergence structure is optionally formed on the sleeve body for diverging light emitted from the plurality of light emitting components, and the supporting body optionally includes a supporting component and a heat dissipating component for conducting heat generated by the plurality of light emitting components to the supporting component. Therefore, the present invention can not only provide bright and wide illumination but also have anti-fogging function.

### CROSS REFERENCE TO RELATED APPLICATIONS

This application claims the benefit of U.S. Provisional Application No. 63/410,606, filed on Sep. 27, 2022. The content of the application is incorporated herein by reference.

### BACKGROUND OF THE INVENTION

### 1. Field of the Invention

The present invention relates to a laryngoscope, and more specifically, to a laryngoscope with advantages of bright and wide illumination and having anti-fogging function.

### 2. Description of the Prior Art

A laryngoscope is a medical device for checking a patient's throat. In order to facilitate a user to check the patient's throat clearly or allow an image capturing component to capture a clear throat image, a blade of the laryngoscope is usually provided with a light source for illumination. However, the conventional laryngoscope still has a drawback of insufficient and/or narrow illumination. Furthermore, a disposable sleeve disposed outside the blade fogs up easily due to temperature difference between inside and outside air, which causes the image capturing component to fail to capture a clear image.

### SUMMARY OF THE INVENTION

Therefore, it is an objective of the present invention to provide a laryngoscope with advantages of bright and wide illumination and having anti-fogging function for solving the aforementioned problems.

In order to achieve the aforementioned objective, the present invention discloses a laryngoscope. The laryngoscope includes a handle, a blade assembly and a plurality of light emitting components. The blade assembly includes a supporting body and a sleeve body. The supporting body is pivotally connected to the handle. The sleeve body is sleeved outside the supporting body. The plurality of light emitting components are disposed on the supporting body and arranged in an arc shape on a first flat plane.

According to an embodiment of the present invention, at least one light divergence structure is formed on the sleeve body.

According to an embodiment of the present invention, a cross section of the at least one light divergence structure on a second flat plane different from the first flat plane is formed in an arc shape or in a wave shape.

According to an embodiment of the present invention, the supporting body includes a supporting component and a heat dissipating component. The heat dissipating component is at least partially disposed inside the supporting component and abuts against the supporting component, and the plurality of light emitting components are disposed on the heat dissipating component.

According to an embodiment of the present invention, the supporting component includes a first end and a second end opposite to the first end. The first end is formed in a flat shape. The second end is pivotally connected to the handle, and the plurality of light emitting components are located adjacent to the first end of the supporting component.

According to an embodiment of the present invention, an opening is formed on the first end of the supporting component, and the blade assembly further includes a covering body disposed on the first end of the supporting component and at least partially covering the opening.

According to an embodiment of the present invention, at least one positioning portion is formed on the covering body and for positioning at least one of the plurality of light emitting components.

According to an embodiment of the present invention, the at least one positioning portion is a positioning recess or a positioning notch.

According to an embodiment of the present invention, the laryngoscope further includes an image capturing assembly centrally disposed on the heat dissipating component or the covering body and located adjacent to the first end of the supporting component.

According to an embodiment of the present invention, the image capturing assembly includes a circuit board and an image capturing component disposed on the circuit board.

According to an embodiment of the present invention, at least one positioning structure is formed on the heat dissipating component and for positioning the image capturing assembly.

According to an embodiment of the present invention, the at least one positioning structure is a positioning wall.

According to an embodiment of the present invention, at least one positioning structure is formed on the covering body and for positioning the image capturing assembly.

According to an embodiment of the present invention, the at least one positioning structure is a positioning block.

According to an embodiment of the present invention, the blade assembly further includes a fastening component. A through hole structure is formed on the supporting component. A cooperating portion is formed on the at least one positioning structure, and the fastening component passes through the through hole structure and is fastened onto the cooperating portion.

According to an embodiment of the present invention, the laryngoscope further includes a light guiding assembly for guiding light to the light emitting assembly.

According to an embodiment of the present invention, at least one first positioning portion and a second positioning portion are formed on the covering body. The at least one first positioning portion is for positioning at least one of the plurality of light emitting components, and the second positioning portion is for positioning the light guiding assembly.

According to an embodiment of the present invention, the at least one first positioning portion or the second positioning portion is a positioning recess or a positioning notch.

According to an embodiment of the present invention, an extending direction of an optical axis of the light guiding assembly is inclined relative to an extending direction of the first end of the supporting component.

According to an embodiment of the present invention, the laryngoscope further includes a display device, and the display device and the supporting body are pivotally connected to two end portions of the handle, respectively.

In summary, the present invention utilizes the light emitting components arranged in an arc shape and the light divergence structure to increase illumination brightness and broaden illumination coverage. Furthermore, the present invention further utilizes the heat dissipating component to rapidly and evenly conduct heat generated by the plurality of light emitting components to the supporting component for reducing a temperature difference between air inside and outside the sleeve body, so as to prevent the sleeve body from fogging. Therefore, the present invention has enhanced using convenience.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 and FIG. 2 are schematic diagrams of a laryngoscope at different views according to a first embodiment of the present invention.

FIG. 3 and FIG. 4 are partial exploded diagrams of the laryngoscope at different views according to the first embodiment of the present invention.

FIG. 5 is a partial diagram of the laryngoscope according to the first embodiment of the present invention.

FIG. 6 is a partial sectional diagram of the laryngoscope according to the first embodiment of the present invention.

FIG. 7 and FIG. 8 are partial exploded diagrams of a laryngoscope at different views according to a second embodiment of the present invention.

FIG. 9 is a partial diagram of the laryngoscope according to the second embodiment of the present invention.

FIG. 10 is a partial sectional diagram of the laryngoscope according to the second embodiment of the present invention.

### DETAILED DESCRIPTION

In the following detailed description of the preferred embodiments, reference is made to the accompanying drawings which form a part hereof, and in which is shown by way of illustration specific embodiments in which the invention may be practiced. In this regard, directional terminology, such as “top”, “bottom”, “left”, “right”, “front”, “back”, etc., is used with reference to the orientation of the Figure(s) being described. The components of the present invention can be positioned in a number of different orientations. As such, the directional terminology is used for purposes of illustration and is in no way limiting. Accordingly, the drawings and descriptions will be regarded as illustrative in nature and not as restrictive. Also, if not specified, the term “connect” is intended to mean either an indirect or direct mechanical connection. Thus, if a first device is coupled to a second device, that connection may be through a direct mechanical connection, or through an indirect mechanical connection via other devices and connections.

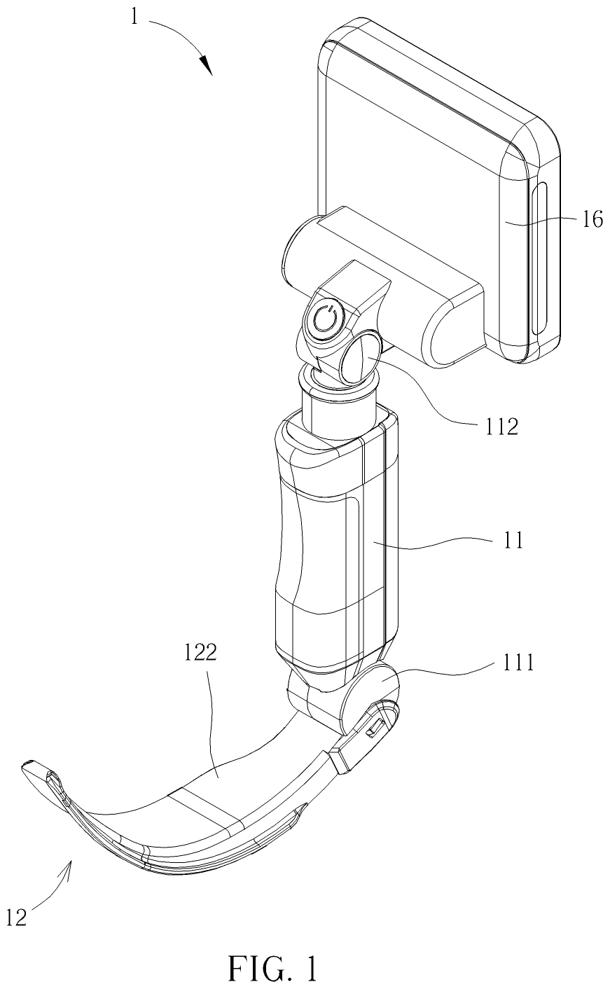

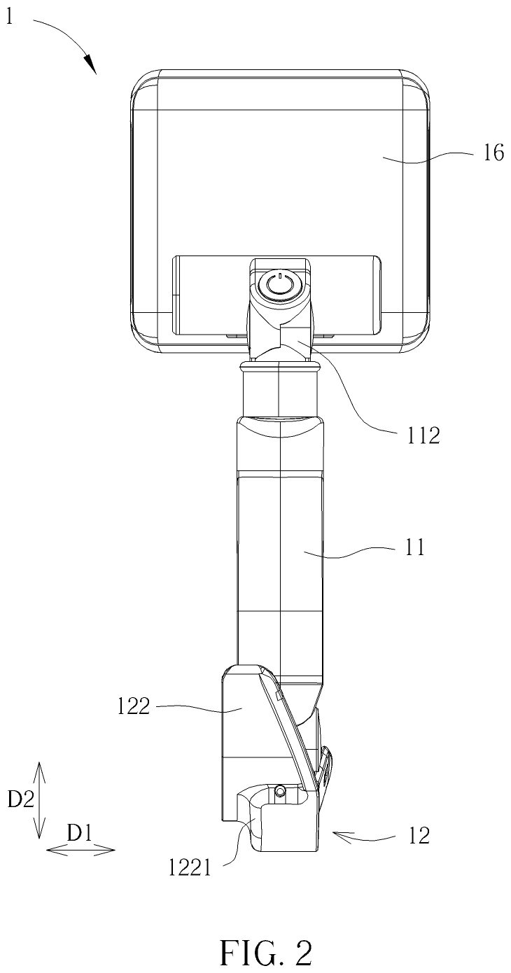

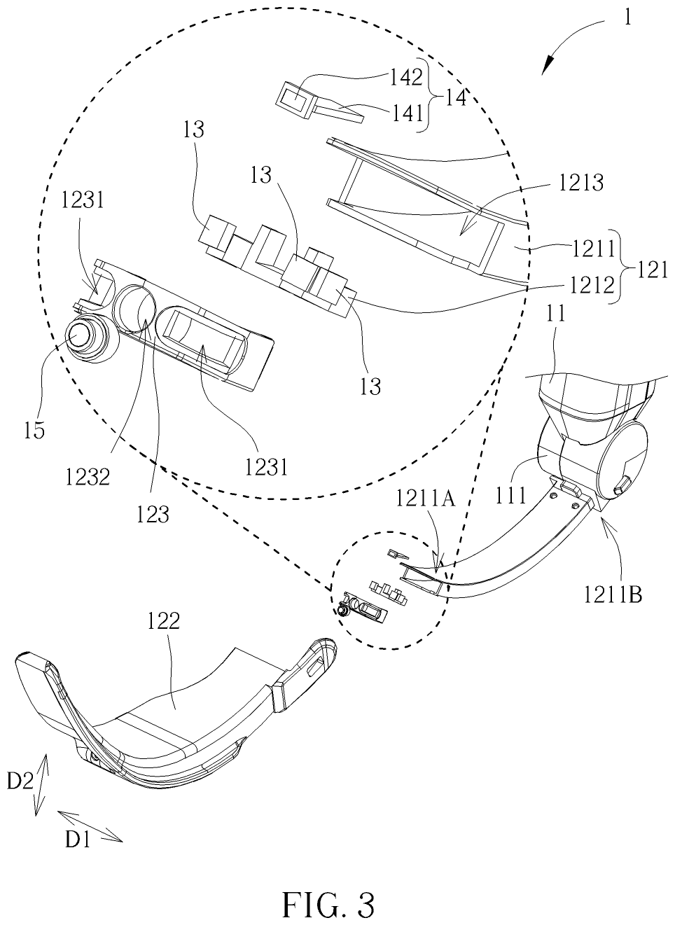

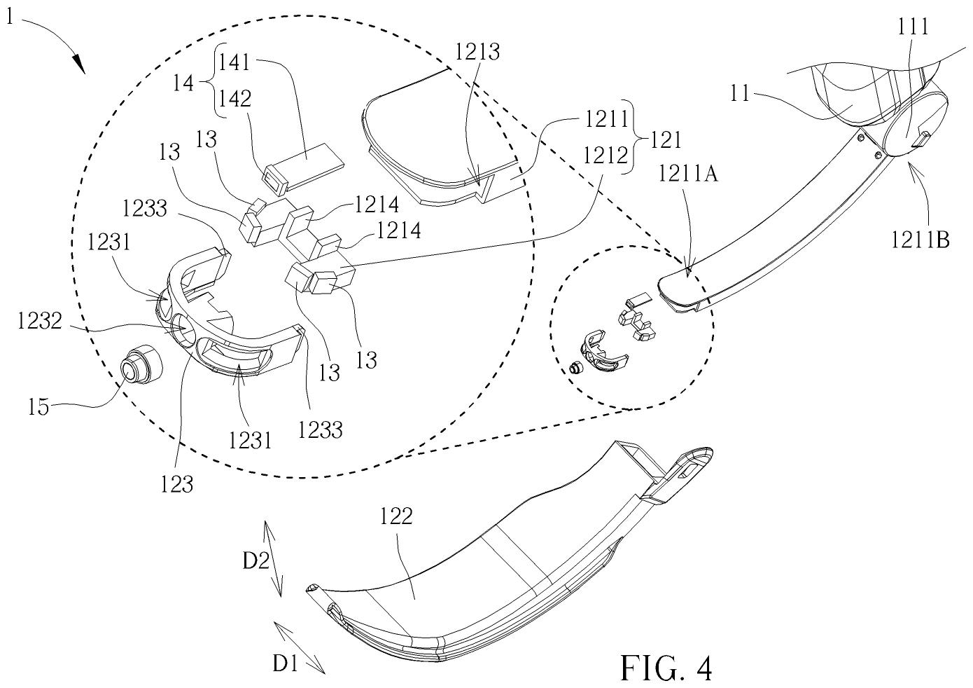

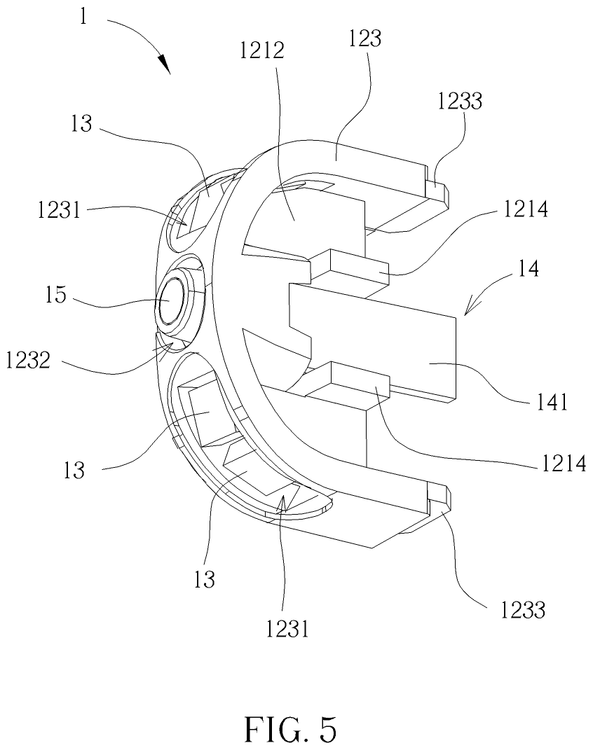

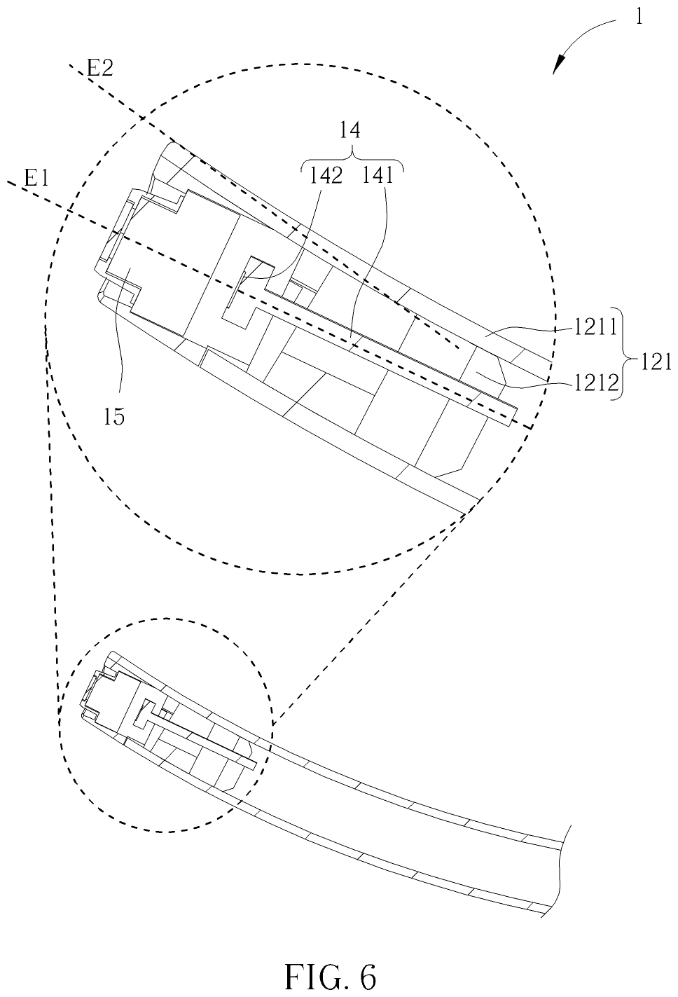

Please refer to FIG. 1 to FIG. 6. FIG. 1 and FIG. 2 are schematic diagrams of a laryngoscope 1 at different views according to a first embodiment of the present invention. FIG. 3 and FIG. 4 are partial exploded diagrams of the laryngoscope 1 at different views according to the first embodiment of the present invention. FIG. 5 is a partial diagram of the laryngoscope 1 according to the first embodiment of the present invention. FIG. 6 is a partial sectional diagram of the laryngoscope 1 according to the first embodiment of the present invention. As shown in FIG. 1 to FIG. 6, the laryngoscope 1 includes a handle 11, a blade assembly 12 and a plurality of light emitting components 13. The blade assembly 12 includes a supporting body 121 and a sleeve body 122. The supporting body 121 is pivotally connected to a first end portion 111 of the handle 11. The sleeve body 122 is sleeved outside the supporting body 121. The plurality of light emitting components 13 are disposed on the supporting body 121 and configured to emit light for providing illumination to a patient's throat when the blade assembly 12 is inserted into the patient's throat.

In this embodiment, the laryngoscope 1 can be a video laryngoscope and further include an image capturing assembly 14, a light guiding assembly 15 and a display device 16. The image capturing assembly 14 is disposed on the supporting body 121 and configured to capture a throat image when the blade assembly 12 is inserted into the patient's throat. The light guiding assembly 15 is configured to guide light to the image capturing assembly 14. The display device 16 is pivotally connected to a second end portion 112 of the handle 11 opposite to the first end portion 111 of the handle 11. The display device 16 can be electrically connected to the image capturing assembly 14, e.g., by an electrical cable. The display device 16 is configured to display the throat image captured by the image capturing assembly 14. However, the present invention is not limited to this embodiment. For example, in another embodiment, the laryngoscope can be a direct laryngoscope and only include the handle, the blade assembly and the light emitting components, and the light capturing assembly, the light guiding assembly and the display device can be omitted.

Specifically, the light emitting component 13 can be a light emitting diode. The image capturing assembly 14 can be a complementary metal-oxide-semiconductor (CMOS) image sensor. The light guiding assembly 15 can be a lens set. However, the present invention is not limited thereto.

In this embodiment, the plurality of light emitting components 13 are arranged in an arc shape along a first direction D1, e.g., a horizontal direction, on a first flat plane, e.g., a horizontal plane. Therefore, the laryngoscope 1 has a wider illumination angle along the first direction D1, e.g., the laryngoscope 1 has a wider horizontal illumination angle.

As shown in FIG. 2, a light divergence structure 1221 is formed on the sleeve body 122. A cross section of the light divergence structure 1221 on a second flat plane different from the first flat plane, whereon the plurality of light emitting components 13 are arranged, is formed in an arc shape.

In this embodiment, since the cross section of the light divergence structure 1221 on the second flat plane, e.g., a vertical flat plane perpendicular to the horizontal flat plane and traversing the sleeve body 122, is formed in the arc shape, the light divergence structure 1221 can diverge the light emitted from the plurality of light emitting components 13 along a second direction different from the first direction D1, e.g., a vertical direction perpendicular to the horizontal direction. Therefore, the laryngoscope 1 has a wider illumination angle along the second direction D2, e.g., the laryngoscope 1 has a wider vertical illumination angle.

Since the laryngoscope 1 has the wider illumination angle along the first direction D1 and the wider illumination angle along the second direction D2, the patient's throat can be well illuminated without any dark zone.

Besides, as shown in FIG. 3 to FIG. 6, the supporting body 121 includes a supporting component 1211 and a heat dissipating component 1212. The supporting component 1211 includes a first end 1211A and a second end 1211B opposite to the first end 1211A. The first end 1211A of the supporting component 1211 is formed in a flat shape, and an opening 1213 is formed on the first end 1211A of the supporting component 1211. The second end 1211B of the supporting component 1211 is pivotally connected to the handle 11. The blade assembly 12 further includes a covering body 123 disposed on the first end 1211A of the supporting component 1211 and at least partially covering the opening 1213. The heat dissipating component 1212 is at least partially disposed inside the supporting component 1211 and abuts against the supporting component 1211. The plurality of light emitting components 13 are disposed on the heat dissipating component 1212 and located adjacent to the first end 1211A of the supporting component 1211. A disposal surface of the heat dissipating component 1212 for arranging the plurality of light emitting components 13 can define the first flat plane. The heat dissipating component 1212 can rapidly and evenly conduct heat generated by the plurality of light emitting components 13 to the supporting component 1211 for reducing a temperature difference between air inside and outside the sleeve body 122, so as to prevent the sleeve body 122 from fogging.

Specifically, the supporting component 1211 can be made of acid and alkali resistance material, e.g., stainless steel. The heat dissipating component 1212 can be made of material with high heat transfer coefficient, e.g., copper. The covering body 123 can be made of transparent or nontransparent plastic material, e.g., polymethyl methacrylate (PMMA). The sleeve body 122 can be made of transparent plastic material, e.g., transparent PMMA. However, the present invention is not limited thereto.

In order to position the plurality of light emitting components 13 and the light guiding assembly 15, two first positioning portions 1231 and a second positioning portion 1232 are formed on the covering body 123. The two first positioning portions 1231 are for positioning the corresponding light emitting components 13 respectively. The second positioning portion 1232 is for positioning the light guiding assembly 15. Specifically, in this embodiment, the first positioning portion 1231 and the second positioning portion 1232 can be positioning notches. However, the present invention is not limited to this embodiment. For example, in another embodiment, the first positioning portion and/or the second positioning portion can be a positioning recess.

Understandably, in another embodiment, the plurality of light emitting components can be fixed by adhesive, e.g., UV glue, for preventing displacement of the plurality of light emitting components after positioning the plurality of light emitting components with the first positioning portions.

In this embodiment, the covering body 123 is attached on the supporting component 1211 in a detachable engaging manner. Specifically, two engaging lugs 1233 are formed on the covering body 123 for engaging with the supporting component 1211.

Besides, as shown in FIG. 3 to FIG. 6, the image capturing assembly 14 includes a circuit board 141 and an image capturing component 142 disposed on the circuit board 141. The light guiding assembly 15 can guide the light to the light capturing component 142. An extending direction E1 of an optical axis of the light guiding assembly 15 is inclined relative to an extending direction E2 of the first end 1211A of the supporting component 1211, i.e., a tangential direction of a distal segment of the first end 1211A, for preventing the image capturing component 142 from receiving light reflected from the supporting component 1211.

In order to position the image capturing assembly 14, as shown in FIG. 5, two positioning structures 1214 are formed on the heat dissipating component 1212 and for positioning the circuit board 141 of the image capturing assembly 14. Specifically, the positioning structure 1214 is a positioning wall. However, the present invention is not limited to this embodiment. For example, in another embodiment, there can be only one positioning structure formed on the heat dissipating component, and the positioning structure can be a positioning recess for at least partially accommodating the circuit board of the image capturing assembly.

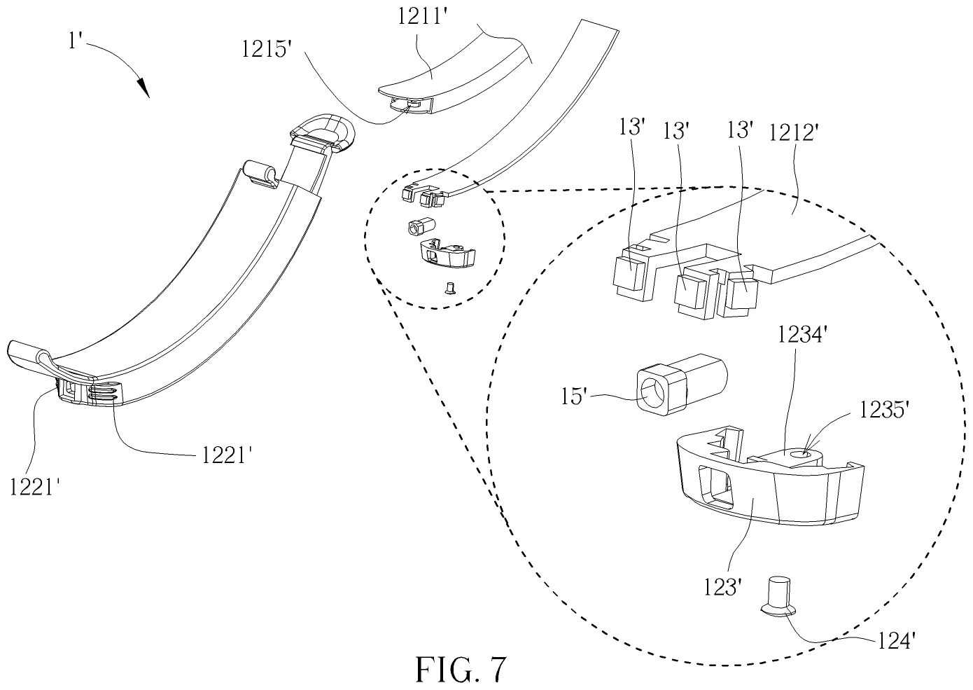

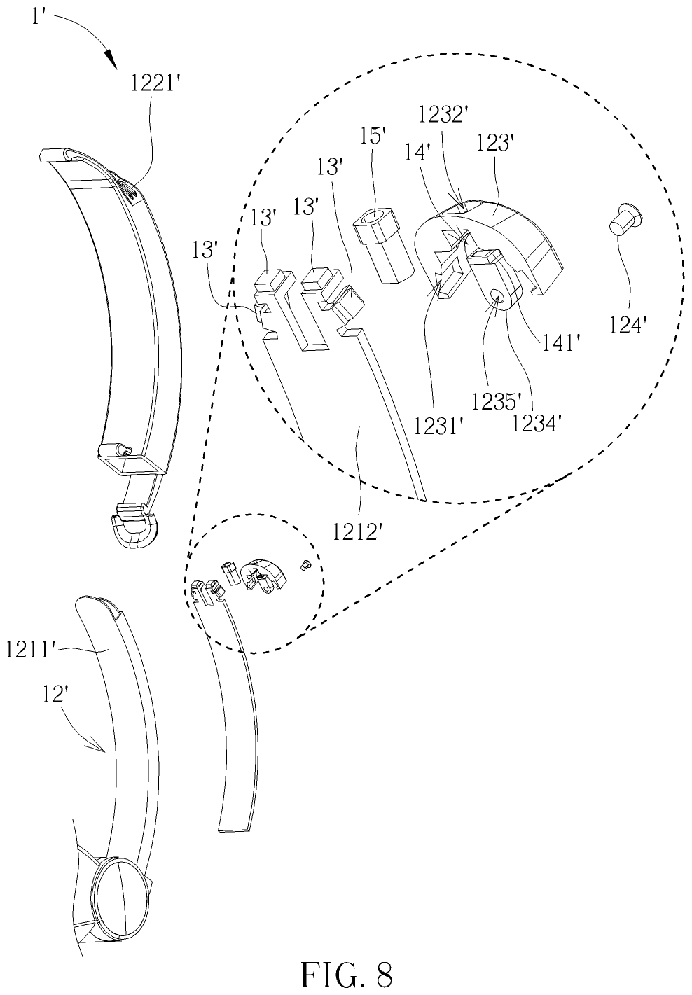

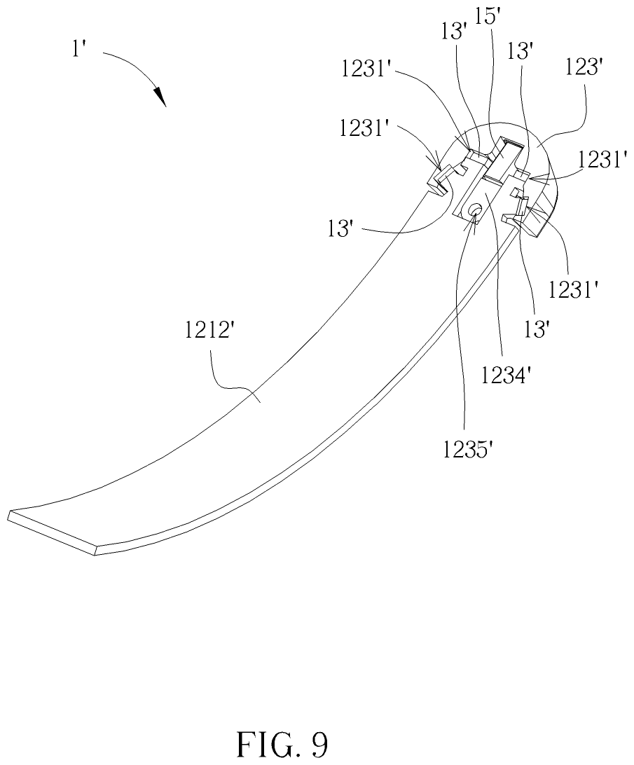

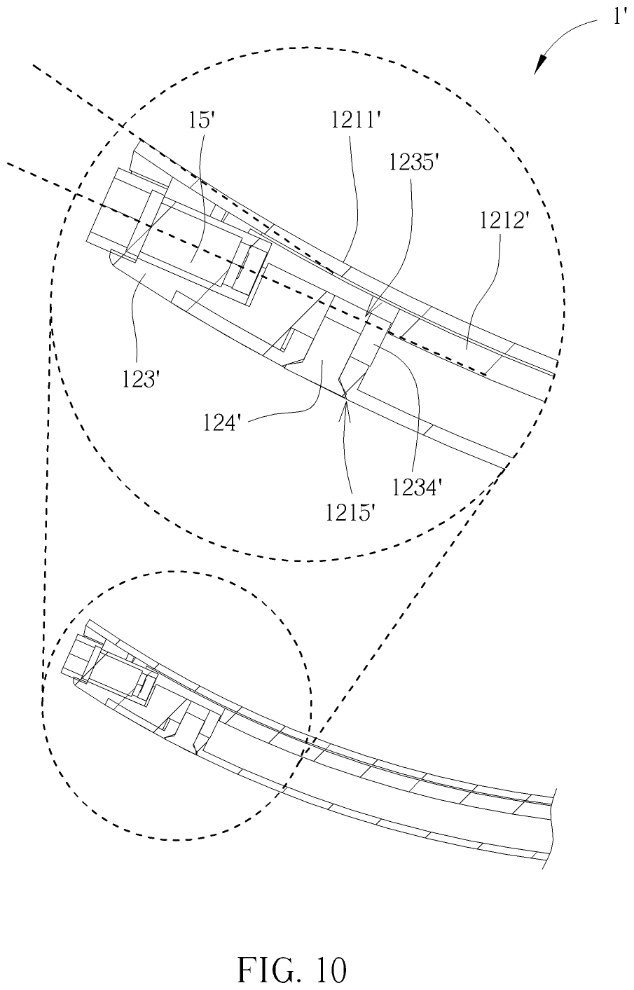

Please refer to FIG. 7 to FIG. 10. FIG. 7 and FIG. 8 are partial exploded diagrams of a laryngoscope 1′ at different views according to a second embodiment of the present invention. FIG. 9 is a partial diagram of the laryngoscope 1′ according to the second embodiment of the present invention. FIG. 10 is a partial sectional diagram of the laryngoscope 1′ according to the second embodiment of the present invention. As shown in FIG. 7 to FIG. 10, different from the first embodiment, a covering body 123′ of a blade assembly 12′ of the laryngoscope 1′ is attached on a supporting body 1211′ in a fastening manner, and an image capturing assembly 14′ is centrally disposed on the covering body 123′. Specifically, a positioning structure 1234′ is formed on the covering body and for positioning a circuit board 141′ of the image capturing assembly 14′. The blade assembly 12′ further includes a fastening component 124′. A through hole structure 1215′ is formed on the supporting component 1211′. A cooperating portion 1235′ is formed on the positioning structure 1234′. The fastening component 124′ passes through the through hole structure 1215′ and is fastened onto the cooperating portion 1235′. More specifically, the positioning structure 1234′ can be a positioning block. The cooperating portion 1235′ can be a screw hole. The fastening component 124′ can be a bolt or a screw. However, the present invention is not limited to this embodiment. For example, in another embodiment, there can be a plurality of clips formed on the covering body and for clipping the circuit board of the image capturing assembly.

Furthermore, in this embodiment, two light divergence structures 1221′ are formed on a sleeve body 122′ and symmetrical to each other relative to an optical axis of a light guiding assembly 15′. A cross section of each light divergence structure 1221′ on the second surface, e.g., the vertical flat plane perpendicular to the horizontal flat plane and traversing the sleeve body 122′, is formed in a wave shape. A heat dissipating component 1212′ extends from an end of the supporting component 1211′ to another end of the supporting component 1211′ and has a greater heat transfer area. The heat dissipating component 1212′ can conduct the heat to the whole supporting component 1211′ more rapidly and evenly. Therefore, it effectively prevents rapid temperature increase of a region near the light emitting components for reducing a temperature difference between air inside and outside the sleeve body 122′, so as to prevent the sleeve body 122′ from fogging.

Besides, in this embodiment, a plurality of first positioning portions 1231′ and a second positioning portion 1232′ are formed on the covering body 123′. The plurality of first positioning portions 1231′ are for positioning the corresponding light emitting components 13′. The second positioning portion 1232′ is for positioning the light guiding assembly 15′. Specifically, the first positioning portion 1231′ and the second positioning portion 1232′ can be a positioning recess and a positioning notch, respectively.

Other details of the laryngoscope 1′ of this embodiment are similar to the ones of the laryngoscope 1′ of the first embodiment. Detailed description is omitted herein for simplicity.

In contrast to the prior art, the present invention utilizes the light emitting components arranged in an arc shape and the light divergence structure to increase illumination brightness and broaden illumination coverage. Furthermore, the present invention further utilizes the heat dissipating component to rapidly and evenly conduct heat generated by the plurality of light emitting components to the supporting component for reducing a temperature difference between air inside and outside the sleeve body, so as to prevent the sleeve body from fogging. Therefore, the present invention has enhanced using convenience.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A laryngoscope comprising:
a handle;
a blade assembly comprising:
a supporting body pivotally connected to the handle; and
a sleeve body sleeved outside the supporting body; and
a plurality of light emitting components disposed on the supporting body and arranged in an arc shape on a first flat plane.

2. The laryngoscope of claim 1, wherein at least one light divergence structure is formed on the sleeve body.

3. The laryngoscope of claim 2, wherein a cross section of the at least one light divergence structure on a second flat plane different from the first flat plane is formed in an arc shape or in a wave shape.

4. The laryngoscope of claim 1, wherein the supporting body comprises a supporting component and a heat dissipating component, the heat dissipating component is at least partially disposed inside the supporting component and abuts against the supporting component, and the plurality of light emitting components are disposed on the heat dissipating component.

5. The laryngoscope of claim 4, wherein the supporting component comprises a first end and a second end opposite to the first end, the first end is formed in a flat shape, the second end is pivotally connected to the handle, and the plurality of light emitting components are located adjacent to the first end of the supporting component.

6. The laryngoscope of claim 5, wherein an opening is formed on the first end of the supporting component, and the blade assembly further comprises a covering body disposed on the first end of the supporting component and at least partially covering the opening.

7. The laryngoscope of claim 6, wherein at least one positioning portion is formed on the covering body and for positioning at least one of the plurality of light emitting components.

8. The laryngoscope of claim 7, wherein the at least one positioning portion is a positioning recess or a positioning notch.

9. The laryngoscope of claim 6, further comprising an image capturing assembly centrally disposed on the heat dissipating component or the covering body and located adjacent to the first end of the supporting component.

10. The laryngoscope of claim 9, wherein the image capturing assembly comprises a circuit board and an image capturing component disposed on the circuit board.

11. The laryngoscope of claim 9, wherein at least one positioning structure is formed on the heat dissipating component and for positioning the image capturing assembly.

12. The laryngoscope of claim 11, wherein the at least one positioning structure is a positioning wall.

13. The laryngoscope of claim 9, wherein at least one positioning structure is formed on the covering body and for positioning the image capturing assembly.

14. The laryngoscope of claim 13, wherein the at least one positioning structure is a positioning block.

15. The laryngoscope of claim 13, wherein the blade assembly further comprises a fastening component, a through hole structure is formed on the supporting component, a cooperating portion is formed on the at least one positioning structure, and the fastening component passes through the through hole structure and is fastened onto the cooperating portion.

16. The laryngoscope of claim 9, further comprising a light guiding assembly for guiding light to the light emitting assembly.

17. The laryngoscope of claim 16, wherein at least one first positioning portion and a second positioning portion are formed on the covering body, the at least one first positioning portion is for positioning at least one of the plurality of light emitting components, and the second positioning portion is for positioning the light guiding assembly.

18. The laryngoscope of claim 17, wherein the at least one first positioning portion or the second positioning portion is a positioning recess or a positioning notch.

19. The laryngoscope of claim 16, wherein an extending direction of an optical axis of the light guiding assembly is inclined relative to an extending direction of the first end of the supporting component.

20. The laryngoscope of claim 1, further comprising a display device, and the display device and the supporting body being pivotally connected to two end portions of the handle, respectively.
